(19) United States
(12) Patent Application Publication
(10) Pub. No.: US 2005/0059824 A1
(43) Pub. Date: Mar. 17, 2005

(54) Method for catalyzing amidation reactions

(75) Inventors: Rajappa Vaidyanathan, Portage, MI (US); Vikram Gurudath Kalthod, Mattawan, MI (US)

Correspondence Address: AGOURON PHARMACEUTICALS, INC., 10350 NORTH TORREY PINES ROAD, LA JOLLA, CA, 92037, US

(73) Assignee: Pharmacia & Upjohn Company

(21) Appl. No.: 10/938,777

(22) Filed: Sep. 9, 2004

Related U.S. Application Data
(60) Provisional application No. 60/501,994, filed on Sep. 11, 2003.

(51) Int. Cl. C07D 279/12; C07D 265/30; C07D 263/34; C07D 213/46

(52) U.S. Cl. 544/59; 544/172; 544/386; 546/315; 548/194; 548/236; 548/530; 562/450; 562/459

(57) ABSTRACT

The invention provides a method of preparing a compound of formula 2 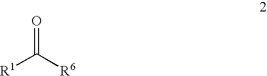
wherein R¹ and R⁶ are as defined herein, by a catalytic amidation process in the presence of added carbon dioxide. The inventive methods show surprising rate enhancement relative to the corresponding uncatalyzed reaction.

This application claims the benefit of U.S. Provisional Application No. 60/501,994, filed Sep. 11, 2003, the disclosure of which is incorporated herein by reference in its entirety.

### FIELD OF THE INVENTION

This invention relates to methods of catalyzing amidation reactions. The inventive methods are particularly useful in catalyzing the reaction of imidazolides with amines to form amides, which can be further reacted to form indolinone compounds that are useful in the treatment of abnormal cell growth, such as cancer, in mammals.

### BACKGROUND

Amides can be prepared by reacting a carboxylic acid substrate with an amine to form the corresponding amide. It is often convenient to replace the hydroxyl moiety of the carboxylic acid with a suitable leaving group R to form a —C(O)R moiety, or use a starting substrate having a —C(O)R moiety rather than an acid group, and react this —C(O)R containing species with an amine to form the amide. Such amidation reactions, however, can be unexpectedly and disadvantageously slow compared to the reactions starting with the corresponding carboxylic acids. Thus, there is a need for methods to increase the rates of amide formation from substrates having —C(O)R moieties, where R is a leaving group.

### SUMMARY

In one embodiment, the present invention provides a method of preparing a compound of formula 2 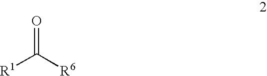
wherein R¹ is selected from the group consisting of C₁₋₂ alkyl, C₃₋₁₂ cycloalkyl, C₂₋₁₂ heterocyclic and C₆₋₁₂ aryl, and R¹ is optionally substituted by from 1 to 6 R³ groups;
each R³ is independently selected from the group consisting of C₆₋₁₂ alkyl, C₁₋₁₂ alkoxy, C₃₋₁₂ cycloalkyl, C₆₋₁₂ aryl, C₂₋₁₂ heterocyclic group containing 1 to 3 atoms selected from N, S and O, C₁₋₁₂ aryloxy, C₆₋₁₂ alkaryl, C₆₋₁₂ alkaryloxy, halogen, trihalomethyl, —S(O)R⁴, —SO₂NR⁴R⁵, —SO₃R⁴, —SR⁴, —NO₂, —NR⁴R⁵, —OH, —CN, —C(O)R⁴, —OC(O)R⁴, —NR⁴C(O)R⁵, —(CH₂)ₙCO₂R⁴, and —CONR⁴R⁵;
R⁴ and R⁵ are independently selected from the group consisting of hydrogen, C₁₋₁₂ alkyl, C₁₋₁₂ cyanoalkyl, C₆₋₁₂ cycloalkyl, C₆₋₁₂ aryl, C₂₋₁₂ heterocyclic group containing 1 to 3 atoms selected from N, S and O, or in the group —NR⁴R⁵, R⁴ and R⁵ may be combined to form a four-, five- or six-membered heterocyclic group optionally containing 1 to 3 atoms selected from N, O, or S in addition to the nitrogen atom to which R⁴ and R⁵ are bound;
R⁶ is selected from —NR⁸(CH₂)ₘR⁹ and —NR¹⁰R¹¹, provided that optionally one to two of the CH₂ groups may be substituted by —OH or halogen;
R⁵ is hydrogen or C₁₋₂ alkyl;
R⁹ is selected from the group consisting of —NR¹⁰R¹¹, —OH, —C(O)R¹², C₆₋₁₂ aryl, C₆₋₁₂ alkaryl, C₆₋₁₂ aryloxy, C₆₋₁₂ alkaryloxy, C₁₋₁₂ alkoxy, C₂₋₁₂ heterocyclic group containing 1 to 3 atoms selected from N, S and O, —N⁺(OR¹⁰, and —NHC(O)R¹³;
R¹⁰ and R¹¹ are independently selected from the group consisting of hydrogen, C₁₋₁₂ alkyl, C₁₋₁₂ cyanoalkyl, C₃₋₁₂ cycloalkyl, C₆₋₁₂ aryl, and C₂₋₁₂ heterocyclic group containing 1 to 3 atoms selected from N, S or O; or R¹⁰ and R¹¹ may be combined to form a four-, five- or six-membered heterocyclic group optionally containing 1 to 3 atoms selected from N, O, or S in addition to the nitrogen atom to which R¹⁰ and R¹¹ are bound, provided that the heterocyclic group formed by R¹⁰ and R¹¹ may optionally be substituted by R⁴;
R¹² is selected from the group consisting of —OH, C₁₋₂ alkoxy, C₆₋₁₂ alkaryl and C₆₋₁₂ aryloxy;
   R¹³ is selected from the group consisting of C₁₋₁₂ alkyl, C₁₋₁₂ haloalkyl, and C₆₋₁₂ aralkyl; n
   is 0, 1 or 2; and
   m is 1, 2, 3 or 4,
      the method comprising reacting a compound of formula 1 with a compound of formula 3 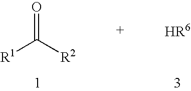
      wherein R¹ and R⁶ are as defined above, and R² is selected from the group consisting of 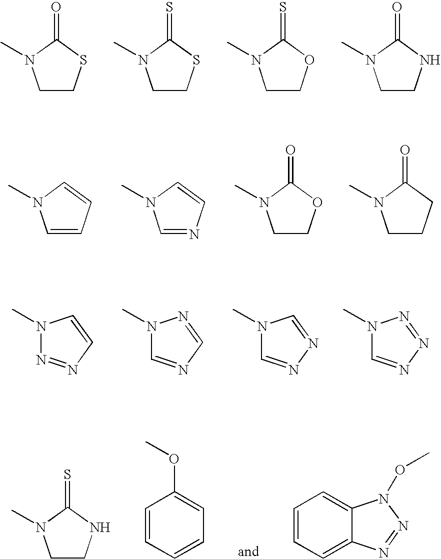
      and R² is optionally substituted by 1 to 6 groups independently selected from the group consisting of halogen, C₁₋₆ alkyl, C₁₋₆ alkoxy, C₆₋₁₂ aryl, C₆₋₁₂ aryloxy, C₆₋₁₂ alkaryl, —NHC(O)R¹⁴ and —C(O)OR¹⁴, where R¹⁴ is hydrogen or a C₁₋₆ alkyl, in the presence of added CO₂, to form the compound of formula 2.

In a specific aspect of this embodiment, R¹ is substituted by at least one R³ group of formula —C(O)R⁴, R⁶ is —NH(CH₂)ₘR⁹ or —NHR¹¹, and the step of reacting the compound of formula 1 with the compound of formula 3 comprises:
(i) forming an intermediate of formula 4 if R⁶ is —NH(CH₂)ₘR⁹ or of formula 5 if R⁶ is NHR¹¹ 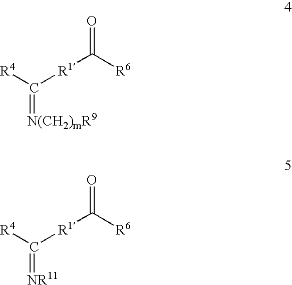
   wherein R¹¹ represents the R¹ moiety without the at least one R³ group of formula —C(O)R⁴; and
(ii) hydrolyzing the imine moiety of the intermediate to form the compound of formula 2.

In another specific aspect of this embodiment, R¹ has the formula 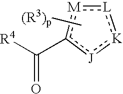
wherein J is selected from the group consisting of O, S and NH;
one of K, L and M is C and the group —C(O)R⁶ is bound thereto, and the others of K, L and M are independently selected from the group consisting of CR³, CR³₂, N, NR³, O and S; and
p is 0, 1 or 2.

In this specific aspect, preferably the 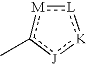
moiety is selected from the group consisting of

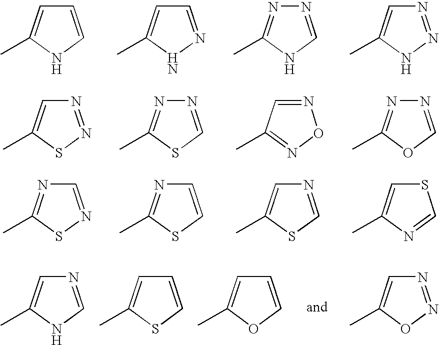

In another specific aspect of this embodiment, the compound of formula 2 is selected from the group consisting of

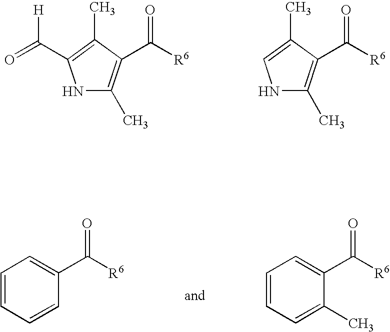

In another specific aspect of this embodiment, R⁶ is —NH(CH₂)ₘR⁹, and R⁹ is selected from the group consisting of —NR¹⁰R¹¹, C₆₋₁₂ aryl, and C₂₋₁₂ heterocyclic group containing 1 to 3 atoms selected from N, S and O.

In another specific aspect of this embodiment, R⁶ is selected from the group consisting of —NHCH₂CH₂N(CH₂CH₃)₂, —NHCH₂CH₂NHCH₂CH₃, —NHCH₂CH₂NH₂ and —NHCH₂(C₆H₅).

In a further aspect of this embodiment, the method further comprises reacting the compound of formula 2 with a compound of formula 6 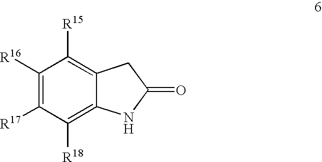
wherein R¹⁵, R¹⁶, R¹⁷ and R¹⁸ are independently selected from the group consisting of hydrogen, C₁₋₁₂ alkyl, C₁₋₁₂ alkoxy, C₃₋₁₂ cycloalkyl, C₆₋₁₂ aryl, C₂₋₁₂ heterocyclic group containing 1 to 3 atoms selected from N, S and O, C₆₋₁₂ aryloxy, C₆₋₁₂ alkaryl, C₆₋₁₂ alkaryloxy, halogen, trihalomethyl, —(O)R⁴, O₂NR⁴R⁵, O₃R⁴, —SR⁴, —NO₂, —NR⁴R⁵, —OH, —CN, —C(O)R⁴, —OC(O)R⁴, —NHC(O)R⁴, —CH₂)ₙCO₂R⁴, and —CONR⁴R⁵
to form a compound of formula 7

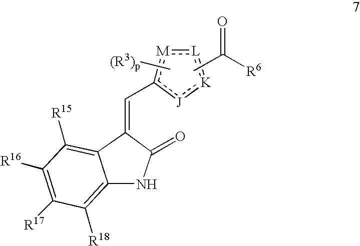

In a specific aspect of this embodiment, the compound of formula 7 is selected from the group consisting of

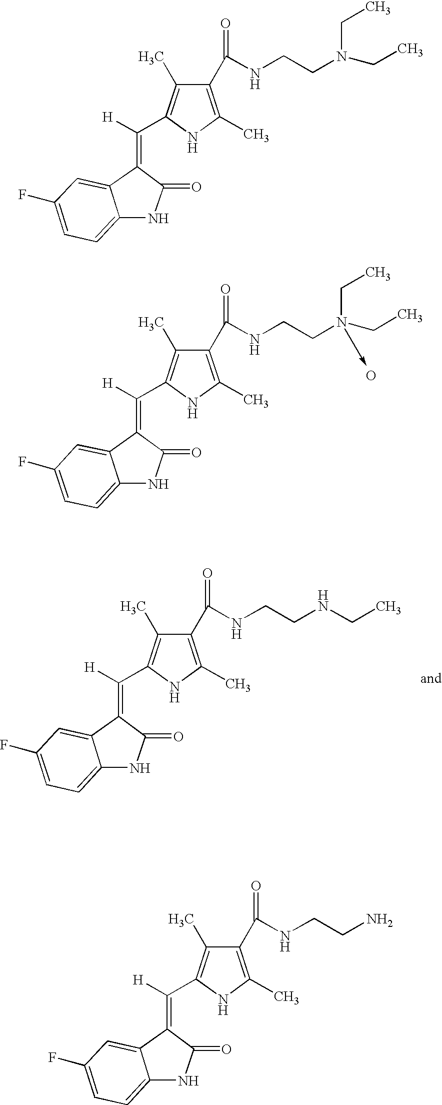

In a specific aspect of this embodiment, the amount of CO₂ added is effective to decrease the reaction time t_{1/2} of the compound of formula 1 with the compound of formula 3 to no more than 75%, preferably no more than 60%, more preferably no more than 50%, of the reaction time t_{1/2} of the corresponding reaction in the absence of added CO₂. As used herein, the term t_{1/2} indicates the amount of time necessary for the reaction to reach 50% completion.

In another specific aspect of this embodiment, the reaction of the compound of formula 1 with the compound of formula 3 is carried out in at least one solvent, and at least a portion of the added CO₂ is provided by introducing CO₂ into the solvent. In this aspect, the CO₂ can be introduced into the neat solvent or into the solvent containing one or both of compounds 1 and 3.

In another embodiment, the present invention provides a method of preparing a compound of formula 8 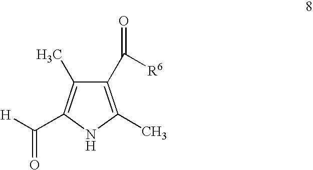
wherein
R⁶ is selected from —NH(CH₂)ₘR⁹ and —NHR¹¹, provided that optionally one to two of the CH₂ groups may be substituted by —OH or halogen;
R⁹ is selected from the group consisting of —NR¹⁰R¹¹, —OH, —C(O)R¹², C₆₋₁₂ aryl, C₆₋₁₂ alkaryl, C₆₋₁₂ aryloxy, C₆₋₁₂ alkaryloxy, C₁₋₁₂ alkoxy, C₂₋₁₂ heterocyclic group containing 1 to 3 atoms selected from N, S and O, —N+(O)R¹⁰, and —NHC(O)R¹³;
   R¹⁰ and R¹¹ are independently selected from the group consisting of hydrogen, C₁₋₁₂ alkyl, C₁₋₁₂ cyanoalkyl, C₃₋₁₂ cycloalkyl, C₆₋₁₂ aryl, and C₂₋₁₂ heterocyclic group containing 1 to 3 atoms selected from N, S or O; or R¹⁰ and R¹¹ may be combined to form a four-, five- or six-membered heterocyclic group optionally containing 1 to 3 atoms selected from N, O, or S in addition to the nitrogen atom to which R¹⁰ and R¹¹ are bound, provided that the heterocyclic group formed by R¹⁰ and R¹¹ may optionally be substituted by a C₁₋₁₂ alkyl, C₁₋₁₂ cyanoalkyl, Cr₅₋₁₂ cycloalkyl, C₆₋₁₂ aryl, or a C₂₋₁₂ heterocyclic group containing 1 to 3 atoms selected from N, S and O;
   R¹² is selected from the group consisting of —OH, C₁₋₁₂ alkoxy, C₆₋₁₂ alkaryl and C₆₋₁₂ aryloxy;
   R¹³ is selected from the group consisting of C₁₋₂ alkyl, C₁₋₂ haloalkyl, and C₆₋₁₂ aralkyl; and
   m is 1, 2, 3 or 4,
      the method comprising reacting a compound of formula 9 with a compound of formula 3 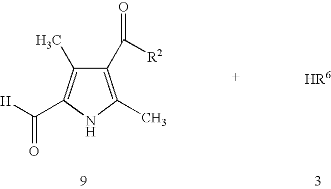
      and R² is selected from the group consisting of 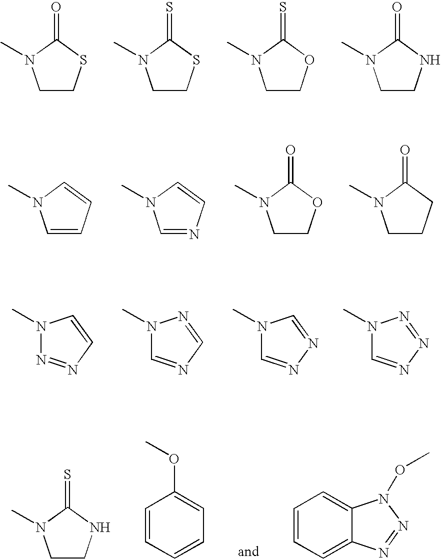
      and R² is optionally substituted by 1 to 6 groups independently selected from the group consisting of halogen, C₁₋₆ alkyl, C₁ alkoxy, C₆₋₁₂ aryl, C₆₋₁₂ aryloxy, C₁₋₁₂ alkaryl, —NHC(O)R¹⁴ and —C(O)OR¹⁴, where R¹⁴ is hydrogen or a C₁₋₆ alkyl, in the presence of added CO₂, to form the compound of formula 8.

In a specific aspect of this embodiment, the step of reacting the compound of formula 9 with the compound of formula 3 comprises:
(i) forming an intermediate of formula 10 if R⁶ is —NH(CH₂)ₘR⁹ or of formula 11 if R⁶ is NHR¹¹ 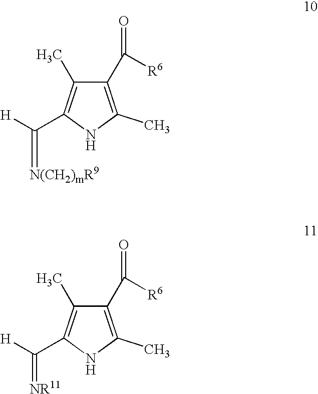
   and
(ii) hydrolyzing the imine moiety of the intermediate to form the compound of formula 8.

In another specific aspect of this embodiment, R⁶ is selected from the group consisting of —NHCH₂CH₂N(CH₂CH₃)₂, —NHCH₂CH₂NHCH₂CH₃, —NHCH₂CH₂NH₂ and —NHCH₂(C₆H₅).

In another specific aspect of this embodiment, the method further comprises reacting the compound of formula 8, 10 or 11 with a compound of formula 6 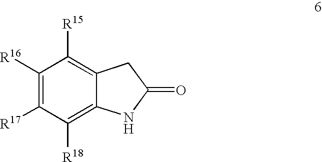
wherein R¹⁵, R¹⁶, R¹⁷ and R¹⁸ are independently selected from the group consisting of hydrogen, C₁₋₁₂ alkyl, C₁₋₂ alkoxy, C₃₋₁₂ cycloalkyl, C₆₋₁₂ aryl, C₂₋₁₂ heterocyclic group containing 1 to 3 atoms selected from N, S and O, C₁₋₁₂ aryloxy, C₆₋₁₂ alkaryl, C₆₋₁₂ alkaryloxy, halogen, trihalomethyl, —S(O)R⁴, —SO₂NR⁴R⁵, —SO₃R⁴, —SR⁴, —NO₂, —NR⁴R⁵, H, —CN, —C(O)R⁴, —OC(O)R⁴, —NHC(O)R⁴, —(CH₂)ₙCO₂R⁴, and —CONR⁴R⁵;
to form a compound of formula 12

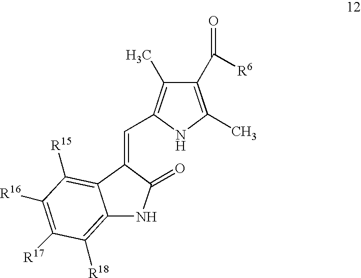

In a specific aspect of this embodiment, the compound of formula 12 is selected from the group consisting of

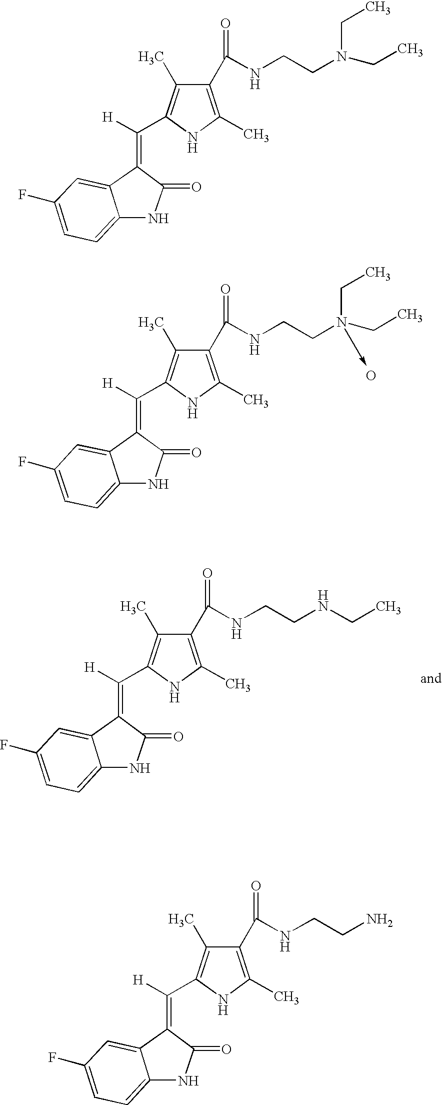

In another specific aspect of this embodiment, the amount of CO₂ added is effective to decrease the reaction time t_{1/2} of the compound of formula 9 with the compound of formula 3 to no more than 75%, preferably no more than 60%, more preferably no more than 50%, of the reaction time t_{1/2} of the corresponding reaction in the absence of added CO₂.

In another specific aspect of this embodiment, the reaction of the compound of formula 1 with the compound of formula 3 is carried out in at least one solvent, and at least a portion of the added CO₂ is provided by introducing CO₂ into the solvent. In this aspect, the CO₂ can be introduced into the neat solvent or into the solvent containing one or both of compounds 9 and 3.

In another embodiment, the present invention provides a method of preparing a compound of formula 13 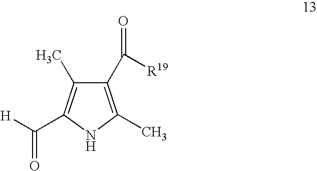
wherein
R¹⁹ is selected from the group consisting of —NHCH₂CH₂N(CH₂CH₃)₂, —NHCH₂CH₂NHCH₂CH₃, —NHCH₂CH₂NH₂ and —NHCH₂(C₆H₅), the method comprising reacting a compound of formula 14 with a compound of formula 15 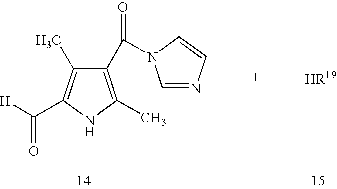
   in the presence of added CO₂, to form the compound of formula 13.

In a specific aspect of this embodiment, the step of reacting the compound of formula 14 with the compound of formula 15 comprises:
(i) forming an intermediate of formula 16 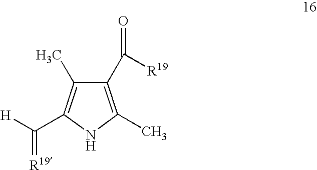
   wherein R^{19′} represents an R¹⁹ group with one nitrogen-bound hydrogen removed to accommodate the imine bond; and
(ii) hydrolyzing the imine moiety of the intermediate to form the compound of formula 13.

In another specific aspect of this embodiment, the method further comprises reacting the compound of formula 13 or 16 with a compound of formula 17 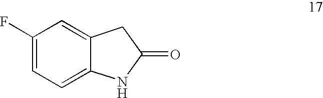
to form a compound of formula 18

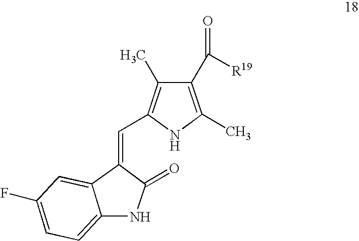

In another specific aspect of this embodiment, the amount of CO₂ added is effective to decrease the reaction time t_{1/2} of the compound of formula 14 with the compound of formula 15 to no more than 75% preferably no more than 60%, more preferably no more than 50%, of the reaction time t_{1/2} of the corresponding reaction in the absence of added CO₂.

In another specific aspect of this embodiment, the reaction of the compound of formula 14 with the compound of formula 15 is carried out in at least one solvent, and at least a portion of the added CO₂ is provided by introducing CO₂ into the solvent. In this aspect, the CO₂ can be introduced into the neat solvent or into the solvent containing one or both of compounds 1 and 3.

In another embodiment, the present invention provides a compound of formula 20 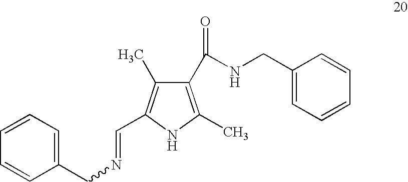
or a salt, preferably a pharmaceutically acceptable salt, or hydrate thereof.

In another embodiment, the present invention provides a compound of formula 21 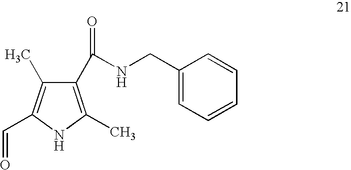
or a salt, preferably a pharmaceutically acceptable salt, or hydrate thereof.

In another embodiment, the present invention provides a compound of formula 22 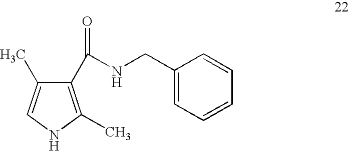
or a salt, preferably a pharmaceutically acceptable salt, or hydrate thereof.

In another embodiment, the present invention provides a compound of formula 23 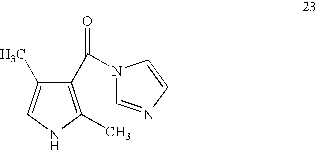
or a salt, preferably a pharmaceutically acceptable salt, or hydrate thereof.

Definitions

The term “halo”, as used herein, unless otherwise indicated, means fluoro, chloro, bromo or iodo. Preferred halo groups are fluoro, chloro and bromo.

The term “alkyl”, as used herein, unless otherwise indicated, includes saturated monovalent hydrocarbon radicals having straight or branched moieties.

The term “alkenyl”, as used herein, unless otherwise indicated, includes alkyl moieties having at least one carbon-carbon double bond wherein alkyl is as defined above and including E and Z isomers of said alkenyl moiety.

The term “alkynyl”, as used herein, unless otherwise indicated, includes alkyl moieties having at least one carbon-carbon triple bond wherein alkyl is as defined above.

The term “alkoxyl”, as used herein, unless otherwise indicated, includes O-alkyl groups wherein alkyl is as defined above.

The term “cycloalkyl”, as used herein, unless otherwise indicated refers to a non-aromatic, saturated or partially saturated, monocyclic or fused, spiro or unfused bicyclic or tricyclic hydrocarbon referred to herein containing a total of from 3 to 10 carbon atoms, preferably 5-8 ring carbon atoms. Exemplary cycloalkyls include monocyclic rings having from 3-7, preferably 3-6, carbon atoms, such as cyclopropyl, cyclobutyl, cyclopentyl, cyclohexyl, cycloheptyl and the like. Illustrative examples of cycloalkyl are derived from, but not limited to, the following:

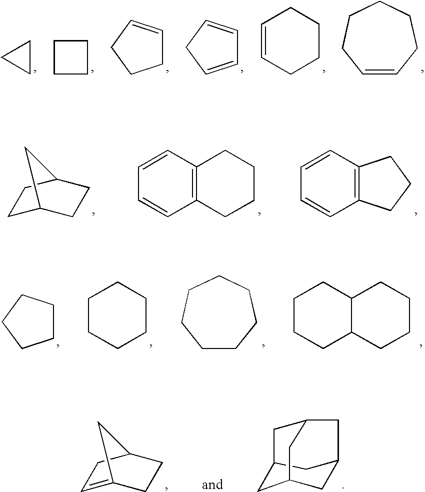

The term “aryl”, as used herein, unless otherwise indicated, includes an organic radical derived from an aromatic hydrocarbon by removal of one hydrogen, such as phenyl or naphthyl.

The term “C₂₋₁₂ heterocyclic”, as used herein, unless otherwise indicated, includes aromatic and non-aromatic heterocyclic groups containing one to three heteroatoms each selected from O, S and N, wherein each heterocyclic group has from 2-12 carbon atoms in its ring system, and with the proviso that the ring of said group does not contain two adjacent O or S atoms. Non-aromatic heterocyclic groups include groups having only 3 atoms in their ring system, but aromatic heterocyclic groups must have at least 5 atoms in their ring system. The heterocyclic groups include benzo-fused ring systems. An example of a 4-membered heterocyclic group is azetidinyl (derived from azetidine). An example of a 5-membered heterocyclic group is thiazolyl and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-2-yl (C-attached). The heterocyclic may be optionally substituted on any ring carbon, sulfur, or nitrogen atom(s) by one to two oxo, per ring. An example of a heterocyclic group wherein 2 ring carbon atoms are substituted with oxo moieties is 1,1-dioxo-thiomorpholinyl. Other Illustrative examples of heterocyclic groups are derived from, but not limited to, the following:

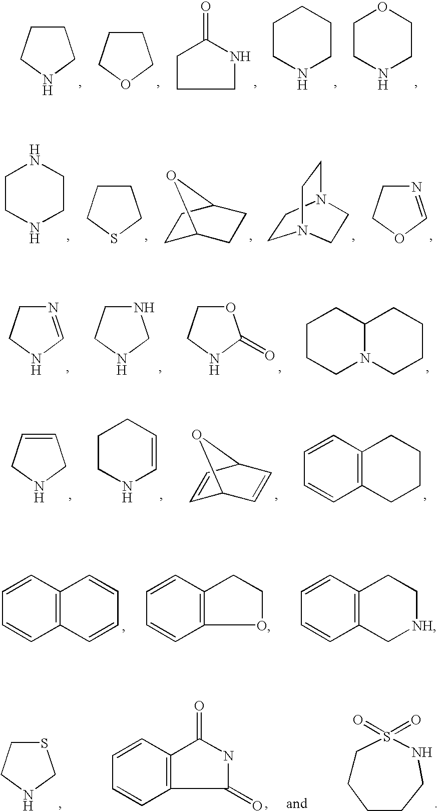

Unless otherwise indicated, the term “oxo” refers to ═O.

The phrase “pharmaceutically acceptable salt(s)”, as used herein, unless otherwise indicated, includes salts of acidic or basic groups which may be present in a compound. Compounds that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phospate/diphosphate, polygalacturonate, salicylate, stearate, subacetate, succinate, tannate, tartrate, teoclate, tosylate, triethiodode, and valerate salts.

### DETAILED DESCRIPTION OF THE INVENTION

The following schemes illustrate the methods and compounds of various embodiments of the present invention. Unless otherwise indicated, the variables used in the reaction schemes and discussion that follows are as defined above.

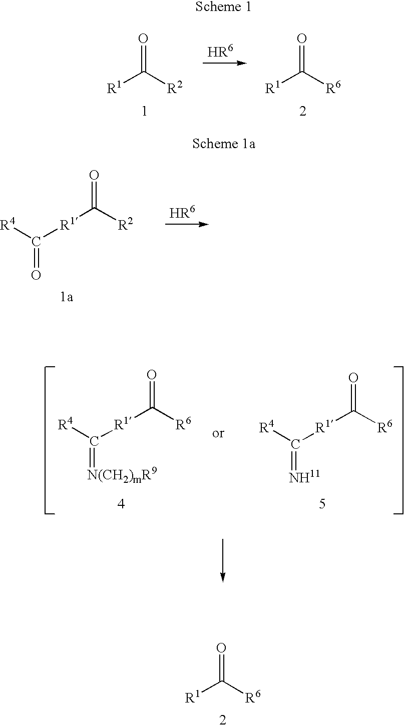

In Scheme 1, a compound of formula 1 having a leaving group R² is reacted with the amine HR⁶, to form the amide compound of formula 2. As shown in Scheme 1a, when the compound of formula 1 includes in the R¹ moiety an aldehyde or ketone group, an intermediate imine-amide 4 or 5 is formed. Under typical HPLC conditions used to monitor the progress of the amidation reaction, the intermediates 4 and 5 are not isolated, but are hydrolyzed to form the amide of formula 2.

Compounds of formula 1 are available commercially, or are readily synthesized from the corresponding carboxylic acids, for example, by reaction of the carboxylic acid with conventional activating agents such as N,N′-carbonyldiimidazole. For example, compounds of formula 1a wherein R¹′ is a heterocyclic group can be obtained by slowly adding POCl₃ to dimethylformamide followed by addition of the appropriate heterocycle, which is also dissolved in dimethylformamide.

This reaction is described in more detail and exemplified, for example, in WO 01/60814, the disclosure of which is incorporated herein by reference.

The reaction of the compound of formula 1 with the compound of formula 3 is generally carried out in a polar aprotic solvent. An aprotic solvent is any solvent that, under normal reaction conditions, does not donate a proton to a solute. Polar solvents are those which have a non-uniform distribution of charge. Generally they include 1 to 3 atoms selected from heteroatom such as N, S or O. Examples of polar aprotic solvents that can be used in the process are ethers such as tetrahydrofuran, diethylether, methyl tert-butyl ether; nitrile solvents such as acetonitrile; and amide solvents such as dimethylformamide. Preferably the reaction solvent is an ether, more preferably the solvent is tetrahydrofuran. Mixtures of solvents may also be used. The aprotic, polar solvent preferably has a boiling point from 30° C. to 130° C., more preferably from 50° C. to 80° C. Both compounds 1 and 3 are introduced into a reaction vessel together with the solvent. The reactants may be added in any order. A reactant concentration of 0.3 to 0.5 mol/L is typical, although one skilled in the art will appreciate that the reaction may be conducted at different concentrations. The reaction may be conducted at a temperature of 0° C. up to the reflux temperature of the solvent. However, it is preferred to conduct the reaction at a temperature of 25° C. to 80° C. with mechanical stirring. The progress of the reaction may be monitored by a suitable analytical method, such as HPLC. The amide 2 may be separated from the reaction mixture by methods known to those skilled in the art, such as, for example, crystallization, extractive workup and chromatography.

Optionally, the compounds of formula 2 having the structure 2a can be further reacted with a compound of formula 6 to form a compound of formula 7, as shown in Scheme 1c.

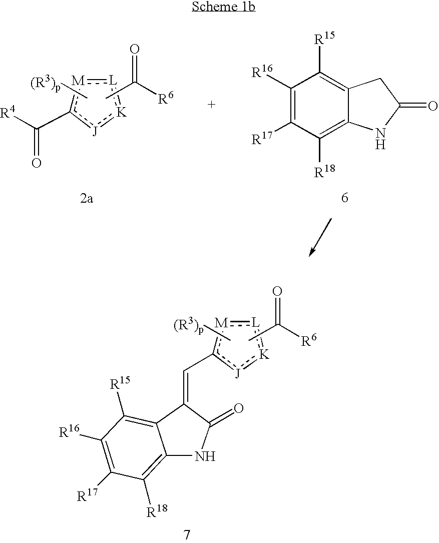

The reaction can be carried out in solution, using the same solvents used in the step of reacting compounds 1 and 3. The reaction may be carried out sequentially by reacting compound 1 with compound 3 and then adding compound 6. However, it is preferred that compounds 1, 3 and 6 are introduced into a reaction vessel together with the solvent. The reactants may be added in any order. A reactant concentration of 0.3 to 0.5 mol/L is typical, although the person of skill in the art will appreciate that the reaction may be conducted at different concentrations. The reaction may be conducted at a temperature of 50° C. up to the reflux temperature of the solvent. However, it is preferred to conduct the reaction at a temperature of 5° C. to 80° C. with mechanical stirring. The progress of the reaction may be monitored by a suitable analytical method, such as HPLC. Compound 7 may be separated from the reaction mixture by methods known to those skilled in the art, such as, for example, crystallization, extractive workup and chromatography. Compound 7 may be further purified by methods known to those skilled in the art, such as recrystallization, if desired.

If desired the compound of formula 7 can be further reacted to form salts or derivatives according to conventional processes.

Schemes 2 and 3 illustrate particular embodiments of the methods of the present invention.

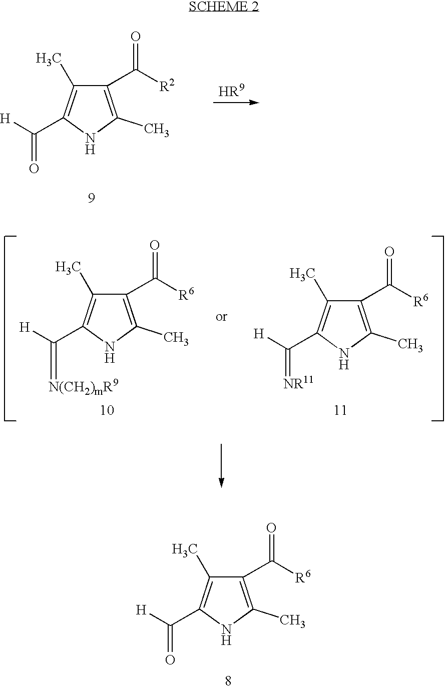

Optionally, the compound of formula 10, 11 or 8 can be further reacted with a compound of formula 6 to form a compound of formula 12, as shown in Scheme 2a starting with a compound of formula 8.

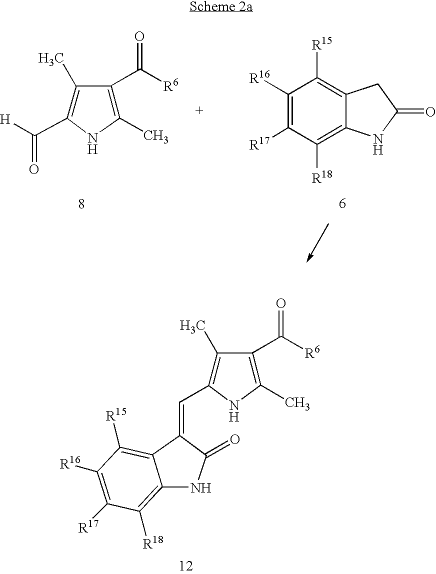

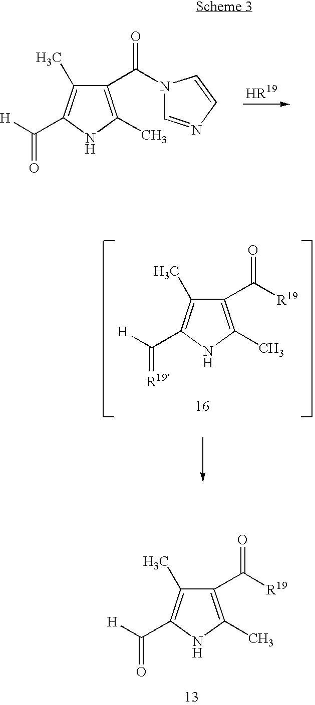

Optionally, the compound of formula 13 or 16 can be further reacted with a compound of formula 17 to form a compound of formula 18, as shown in Scheme 3a, starting with a compound of formula 13.

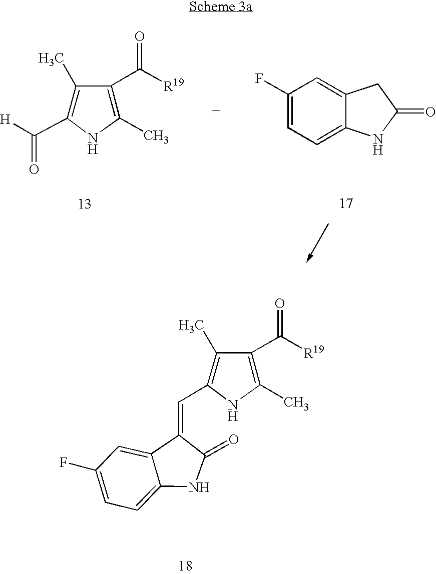

In a particularly preferred aspect of the methods shown in Schemes 1b, 2a and 3a, the method is used to form indolinone compounds of formula 7, 12 and 18, respectively. A number of indolinone derivatives have been found to exhibit pharmaceutical activity. Due to the ability to modulate the protein kinase activity, they have been suggested to treat an number of conditions such as various types of cancer, mastocytosis, allergy associated chronic rhinitis, diabetes, autoimmune disorders, restenosis, fibrosis, psoriasis, von Hippel-Lindau disease, osteoarthritis, rheumatoid arthritis, angiogenesis, inflammatory disorders, immunological disorders, and cardiovascular disorders. Such compounds are described, for example, in U.S. Pat. No. 6,573,293, and in PCT publication Nos. WO 01/37820, published May 31, 2001; WO 01/45689, published Jun. 28, 2001; WO 02/081466, published Oct. 17, 2002; WO 01/090103, published Nov. 29, 2001; WO 01/090104, published Nov. 29, 2001; WO 01/90068, published Nov. 29, 2001; WO 03/015608, published Feb. 27, 2003; WO 03/045307, published Jun. 5, 2003, WO 03/035009, published May 1, 2003; WO 03/016305, published February 27, 2003; and copending U.S. application Ser. No. 10/367,008, filed Feb. 14, 2003. The disclosures of these references are incorporated herein by reference in their entireties.

In particularly preferred embodiments, the compound of formula 7, 12 or 18 is selected from the group consisting of

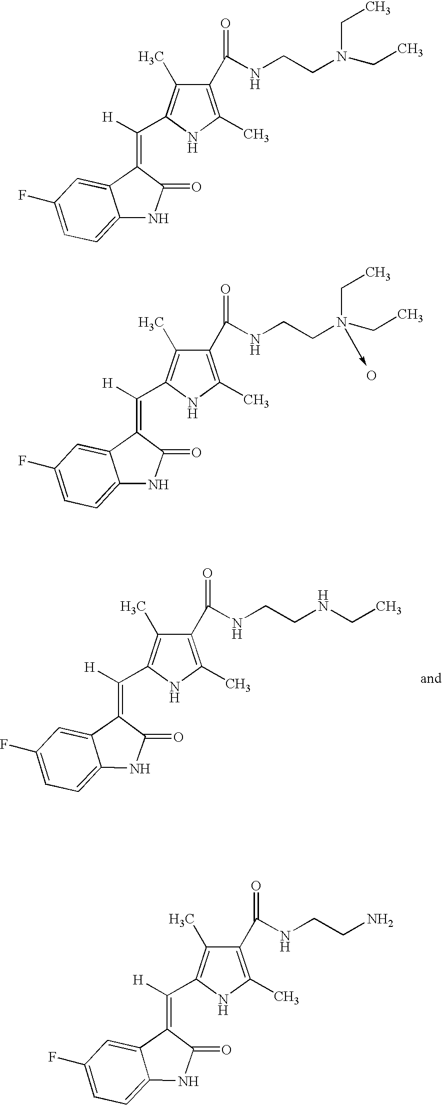

It has been surprisingly found that CO₂ catalyzes the amidation reactions shown in the above-described reaction schemes, significantly increasing the reaction rates. This result is particularly unexpected, as CO₂ catalysis of amidation reactions has not been reported, and CO₂ might be expected to react with the amine to form a carbamate salt, thus slowing down the amidation reaction.

CO₂ can be provided to the reaction by any convenient means. For example, all or part of the CO₂ can be provided to a mixture containing one or more of the reagents and a solvent, or to the neat solvent. The CO₂ can be provided prior to, or at any point during, the reaction in single or multiple aliquots, or continuously. The CO₂ can be bubbled into a solvent or mixture, or the reaction can be carried out under CO₂ pressure, provided that sufficient CO₂ dissolves in the solvent or mixture to be catalytically effective. In a preferred method, CO₂ is bubbled into a mixture of the amine HR⁶ or HR¹⁹ in a solvent, such as THF, for a period of from 1 minute to several hours, preferably for about 15 minutes, and the starting material subsequently added. One skilled in the art can readily determine when sufficient CO₂ is present by monitoring the reaction rate. As the amount of CO₂ provided is increased, the reaction rate reaches a maximum beyond which the provision of additional CO₂ has no effect.

In other embodiments, the invention provides compounds of formulae 20-23. 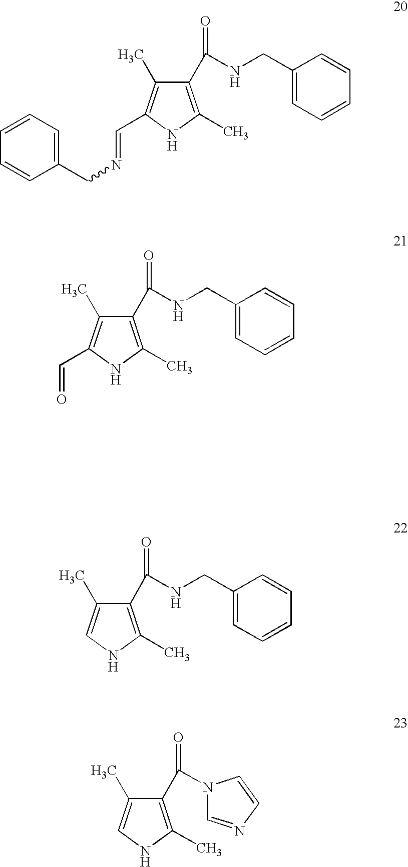
and their salts, preferably pharmaceutically acceptable salts, and hydrates. Compounds 20-23 can be synthesized as shown in the Examples below. The wavy bond between the imine and benzyl moieties indicates that both cis and trans configurations are contemplated.

The compounds of formulas 20-23 are capable of forming a wide variety of different salts with various inorganic and organic acids. Although such salts must be pharmaceutically acceptable for administration to animals, it is often desirable in practice to initially isolate the compounds from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

In the following examples and preparations, “Et” means ethyl, and “Ph” means phenyl.

### EXAMPLES 1-10

The imidazolides shown in Table 1 were prepared from the corresponding carboxylic acids by reaction with N,N′-carbonyldiimidazole. The resulting imidazolide was reacted with the amine shown in Table 1 both with and without the presence of added carbon dioxide. A typical procedure was as follows. A mixture of the carboxylic acid (6 mmol) and N,N′-carbonyldiimidazole (CDI) (7.2 mmol) in tetrahydrofuran (THF) (20 mL) was stirred at 45° C. When HPLC indicated complete conversion to the imidazolide, the mixture was concentrated to dryness in vacuo to remove all CO₂. This mixture containing the imidazolide and imidazole was diluted with 10 mL THF. In a separate flask, CO₂ was bubbled through a solution of the amine (7.8 mmol, 1.3 equiv) in THF (10 mL) for 15 min. This solution was added to the solution of the imidazolide and imidazole, and stirred at 45° C. The reaction was monitored by HPLC. For the uncatalyzed reactions, a solution of the amine in 10 mL THF was added to a solution of the imidazolide and imidazole in 10 mL THF. For Examples 1 and 2, 3 equivalents of amine were added for both the catalyzed and uncatalyzed reactions. The products were characterized by ¹H and ¹³C NMR and compared to literature values.

TABLE 1
summarizes Examples 1-10.

t_{1/2}(min)^{a}

| Example | Imidazolide | Amine | Product | Catalyzed | Uncatalyzed |
|---|---|---|---|---|---|
| 1^{b} | 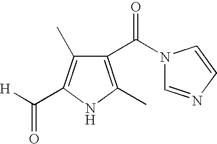 | 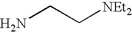 | 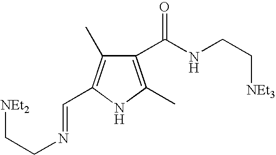 | 53 | >330^{c} |
| 2^{b} | 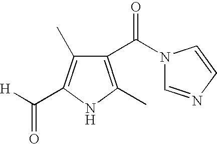 | 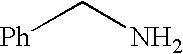 | 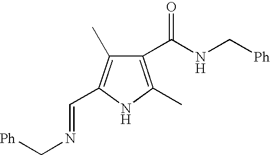 | 101 | 217 |
| 3 | 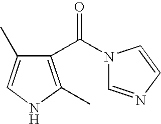 | 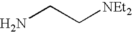 | 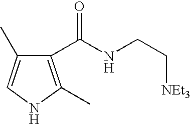 | 98 | 410 |
| 4 | 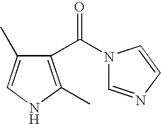 | 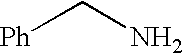 | 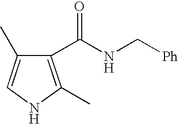 | 161 | >>510^{d} |
| 5 | 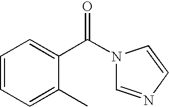 | 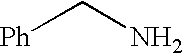 | 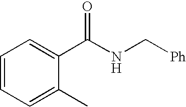 | 11 | 192 |
| 6 | 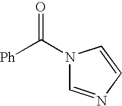 | 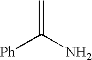 | 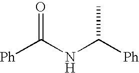 | 13 | >275^{e} |
| 7 | 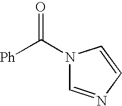 | 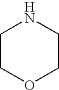 | 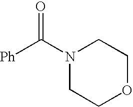 | <15^{f} | 72 |
| 8 | 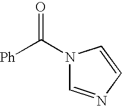 | 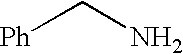 | 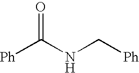 | <1^{g} | <10^{h} |
| 9 | 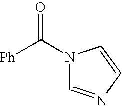 | 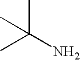 | 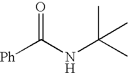 | n.r.ⁱ | n.r.ⁱ |
| 10 | 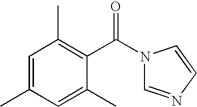 | 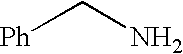 | 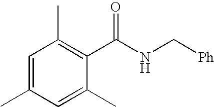 | n.r.ⁱ | n.r.ⁱ |

^{a}t_{1/2} is the time required for the amidation reaction to reach 50% conversion by HPLC.
^{b}The product imine-amides were hydrolyzed to the corresponding aldehyde-amides under the HPLC conditions.
^{c}The reaction was 48% complete in 330 min.
^{d}The reaction was 11% complete in 510 min.
^{e}The reaction was 43% complete in 275 min.
^{f}The reaction was 100% complete in 30 min.
^{g}The reaction was 97% complete in 1 min.
^{h}The reaction was 34% complete in 1 min. and 92% complete in 10 min.
ⁱNo reaction

Compounds of formulae 21-23 were synthesized as follows.

### EXAMPLE 11

N-Benzyl-5-formyl-2,4-di methyl-1H-pyrrole-3-carboxamide

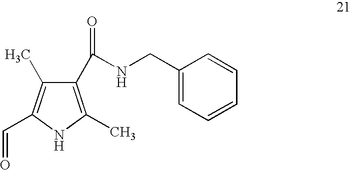

Hydroxybenzotriazole (0.49 g), 1-(3-dimethylaminopropyl)-3-ethylcarbodiimide hydrochloride (7.45 g), triethylamine (5.74 g), benzyl amine (3.20 g) and acetonitrile (30 mL) were added to 500 mL 3-neck round-bottomed flask. The resulting solution was stirred vigorously while 5-formyl-2,4-dimethyl-1H-pyrrole-3-carboxylic acid (5.00 g) in acetonitrile (20 mL) was added to it. The mixture was stirred at room temperature under an atmosphere of N₂ for three hours. After this time, the mixture was diluted with water, brine, saturated NaHCO₃, and the pH adjusted to >10 with 50% NaOH solution. The aqueous mixture was then extracted with a 90% CH₂Cl₂/MeOH (2×250 mL) solution. The organics were dried over sodium sulfate and concentrated giving light orange solids, which were collected by suction filtration and washed with cold acetonitrile. The product was isolated as an off white solid (1.45 g) in 21% yield. ¹H NMR (DMSO-d₆) δ 11.85 (s, 1H), 9.55 (s, 1H), 8.11-8.08 (m, 1H), 7.34-7.22 (m, 4H), 4.42 (d, J=6.1 Hz, 2H), 2.38 (s, 3H), 2.33 (s, 3H). HRMS (ES) found m/z 257.1290 (M+H⁺) C₁₅H₁₆N₂O₂+H requires 257.1295.

### EXAMPLE 12

N-Benzyl-2,4-dimethyl-1H-pyrrole-3-carboxamide

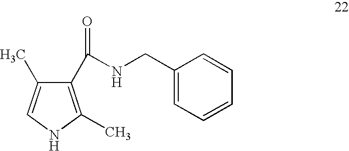

Hydroxybenzotriazole (0.35 g), 1-(3-dimethylaminopropyl)-3-ethylcarbodiimide hydrochloride (5.37 g), triethylamine (4.14 g), benzyl amine (2.31 g), and acetonitrile (20 mL) were added to 250 mL 3-neck round-bottomed flask. The resulting solution was stirred vigorously while 2,4-dimethyl-1H-pyrrole-3-carboxylic acid (3.00 g) in acetonitrile (20 mL) was added to it. The mixture was stirred at room temperature under an atmosphere of N₂ for three hours. After this time, the mixture was diluted with water, brine, saturated NaHCO₃, and the pH adjusted to >10 with 50% NaOH solution. The aqueous mixture was then extracted with 90% CH₂Cl₂/MeOH (2×250 mL). The organics were dried over sodium sulfate and concentrated in vacuo yielding a yellow oil. The crude material was chromatographed (SiO₂; 1% methanol/methylene chloride) to afford 2.05 g (42%) of the product as white crystals. ¹H NMR (DMSO-d₆) δ 10.53 (s, 1H), 7.54 (t, J=6.1 Hz, 1H), 7.33-7.29 (m, 3H), 7.247.21 (m, 1H), 6.33 (s, 1H), 4.40 (d, J=6.0 Hz, 2H), 2.28 (s, 3H), 2.09 (s, 3H). HRMS (ES) found m/z 229.1341 (M+H⁺) C₁₄H₁₆N₂O₁+H requires 229.1332.

### EXAMPLE 13

3-(1H-Imidazol-1-ylcarbonyl)-2,4-dimethyl-1H-pyrrole

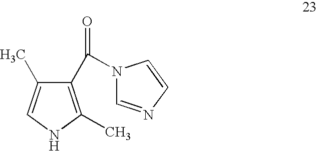

Carbonyldiimidazole (9.73 g), 2,4-dimethyl-1H-pyrrole-3-carboxylic acid (6.96 g) and tetrahydrofuran (150 mL) were combined in a 500 mL round-bottomed flask and stirred at 45° C. for three hours. The solution was concentrated in vacuo, and acetonitrile (25 mL) was added to the residue. The resulting slurry was filtered to afford 7.28 g (77%) of the product. ¹H NMR (DMSO-d₆) δ 11.25 (s, 1H), 8.02 (s, 1H), 7.52 (s, 1H), 7.05 (s, 1H), 6.57 (s, 1H), 2.11 (s, 3H), 1.95 (s, 3H). HRMS (ES) found m/z 190.0980 (M+H⁺) C₁₀H₁₁N₃O+H requires 190.0987.

While the invention has been illustrated by reference to specific and preferred embodiments, those skilled in the art will recognize that variations and modifications may be made through routine experimentation and practice of the invention. Thus, the invention is intended not to be limited by the foregoing description, but to be defined by the appended claims and their equivalents.

1. A method of preparing a compound of formula 2 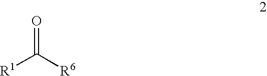
wherein R¹ is selected from the group consisting of C₁₋₁₂ alkyl, C₃₋₁₂ cycloalkyl, C₂₋₁₂ heterocyclic and C₆₋₁₂ aryl, and R¹ is optionally substituted by from 1 to 6 R³ groups;
each R³ is independently selected from the group consisting of C₁₋₁₂ alkyl, C₁₋₁₂ alkoxy, C₃₋₁₂ cycloalkyl, C₆₋₁₂ aryl, C₂₋₁₂ heterocyclic group containing 1 to 3 atoms selected from N, S and O, C₆₋₁₂ aryloxy, C₆₋₁₂ alkaryl, C₆₋₁₂ alkaryloxy, halogen, trihalomethyl, —S(O)R⁴, —SO₂NR⁴R⁵, —SO₃R⁴, —SR⁴, —NO₂, —NR⁴R⁵, —OH, —CN, —C(O)R⁴, —OC(O)R⁴, —NR⁴C(O)R⁵, —(CH₂)ₙCO₂R⁴, and —CONR⁴R⁵;
R⁴ and R⁵ are independently selected from the group consisting of hydrogen, C₁₋₁₂ alkyl, C₁₋₁₂ cyanoalkyl, C₅₋₁₂ cycloalkyl, C₆₋₁₂ aryl, C₂₋₁₂ heterocyclic group containing 1 to 3 atoms selected from N, S and O, or in the group —N R⁴R⁵, R⁴ and R⁵ may be combined to form a four-, five- or six-membered heterocyclic group optionally containing 1 to 3 atoms selected from N, O, or S in addition to the nitrogen atom to which R⁴ and R⁵ are bound;
R⁶ is selected from —NR⁸(CH₂)ₘR⁹ and —NR¹⁰R¹¹, provided that optionally one to two of the CH₂ groups may be substituted by H or halogen;
R⁸ is hydrogen or C₁₋₁₂ alkyl;
R⁹ is selected from the group consisting of —NR¹⁰R¹¹, —OH, —C(O)R², C₁₋₁₂ aryl, C₆₋₁₂ alkaryl, C₆₋₁₂ aryloxy, C₆₋₁₂ alkaryloxy, C₁₋₁₂ alkoxy, C₂₋₁₂ heterocyclic group containing 1 to 3 atoms selected from N, S and O, —N⁺(O⁻)R¹⁰, and —NHC(O)R¹³;
R¹⁰ and R¹¹ are independently selected from the group consisting of hydrogen, C₁₋₁₂ alkyl, C₁₋₁₂ cyanoalkyl, C₃₋₁₂ cycloalkyl, C₆₋₁₂ aryl, and C₂₋₁₂ heterocyclic group containing 1 to 3 atoms selected from N, S or O; or R¹⁰ and R¹¹ may be combined to form a four-, five- or six-membered heterocyclic group optionally containing 1 to 3 atoms selected from N, O, or S in addition to the nitrogen atom to which R¹⁰ and R¹¹ are bound, provided that the heterocyclic group formed by R¹⁰ and R¹¹ may optionally be substituted by R⁴;
R¹² is selected from the group consisting of —OH, C₁₋₁₂ alkoxy, C₆₋₁₂ alkaryl and C₆₋₁₂ aryloxy;
R¹³ is selected from the group consisting of C₁₋₁₂ alkyl, C₁₋₁₂ haloalkyl, and C₆₋₁₂ aralkyl; n is 0, 1 or 2; and
m is 1, 2, 3 or 4,
the method comprising reacting a compound of formula 1 with a compound of formula 3 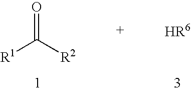
wherein R¹ and R⁶ are as defined above, and R² is selected from the group consisting of 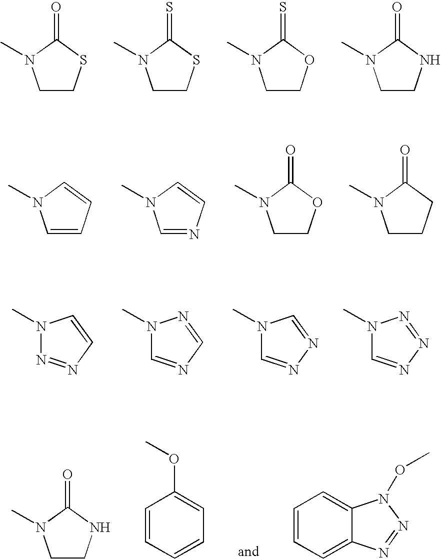
and R² is optionally substituted by 1 to 6 groups independently selected from the group consisting of halogen, C₁₋₄ alkyl, C₁₋₄ alkoxy, C₆₋₁₂ aryl, C₆₋₁₂ aryloxy, C₆₋₁₂ alkaryl, —NHC(O)R¹⁴ and —C(O)OR¹⁴, where R¹⁴ is hydrogen or a C₁₋₆ alkyl, in the presence of added CO₂, to form the compound of formula 2.

2. The method of claim 1, wherein R¹ is substituted by at least one R³ group of formula —C(O)R⁴, R⁶ is —NH(CH₂)ₘR⁹ or —NHR¹¹, and wherein the step of reacting the compound of formula 1 with the compound of formula 3 comprises:
(i) forming an intermediate of formula 4 if R⁶ is —NH(CH₂)ₘR⁹ or of formula 5 if R⁶ is NHR¹¹ 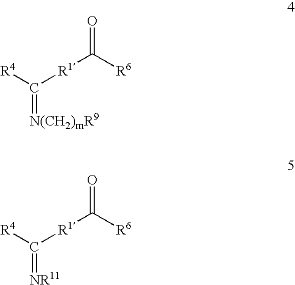
wherein R¹, represents the R¹ moiety without the at least one R³ group of formula —C(O)R⁴; and
(ii) hydrolyzing the imine moiety of the intermediate to form the compound of formula 2.

3. The method of claim 1, wherein R¹ has the formula 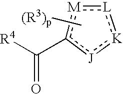
wherein J is selected from the group consisting of O, S and NH;
one of K, L and M is C and the group —C(O)R⁶ is bound thereto, and the others of K, L and M are independently selected from the group consisting of CR³, CR³₂, N, NR³, O and S; and
p is 0, 1 or 2.

4. The method of claim 3, wherein the 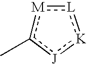
moiety is selected from the group consisting of

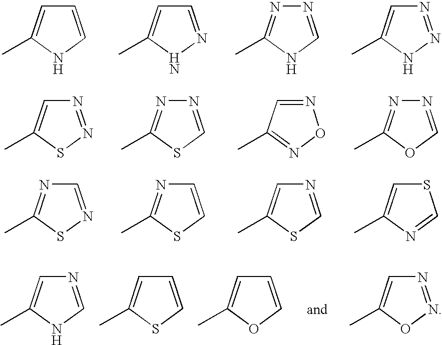

5. The method of claim 1, wherein the compound of formula 2 is selected from the group consisting of

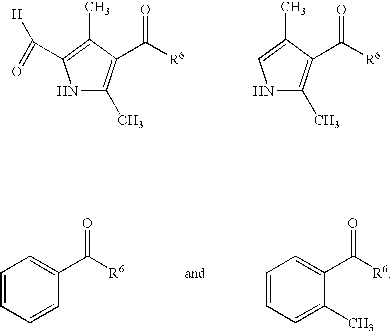

6. The method of claim 1, wherein R⁶ is —NH(CH₂)ₘR⁹, and R⁹ is selected from the group consisting of —NR¹⁰R¹¹, C₆₋₁₂ aryl, and C₂₋₁₂ heterocyclic group containing 1 to 3 atoms selected from N, S and O.

7. The method of claim 1, wherein R⁶ is selected from the group consisting of —NHCH₂CH₂N(CH₂CH₃)₂, —NHCH₂CH₂NHCH₂CH₃, —NHCH₂CH₂NH₂ and —NHCH₂(C₆H₅).

8. The method of claim 3, further comprising reacting the compound of formula 2 with a compound of formula 6 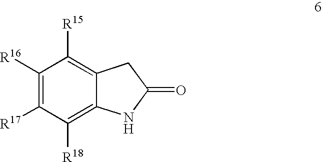
wherein R¹⁵, R¹⁶, R¹⁷ and R¹⁸ are independently selected from the group consisting of hydrogen, C₁₋₁₂ alkyl, C₁₋₁₂ alkoxy, C₃₋₁₂ cycloalkyl, C₆₋₁₂ aryl, C₆₋₁₂ heterocyclic group containing 1 to 3 atoms selected from N, S and O, C₆₋₁₂ aryloxy, C₆₋₁₂ alkaryl, C₆₋₁₂ alkaryloxy, halogen, trihalomethyl, —S(O)R⁴, —SO₂NR⁴R⁵, —SO₃R⁴, —SR⁴, —NO₂, —NR⁴R⁵, H, N, (O)R⁴, OC(O)R⁴, —NHC(O)R⁴, —(CH₂)ₙCO₂R⁴, and —CONR⁴R⁵;
to form a compound of formula 7

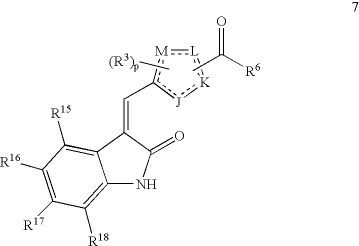

9. The method of claim 8, wherein the compound of formula 7 is selected from the group consisting of

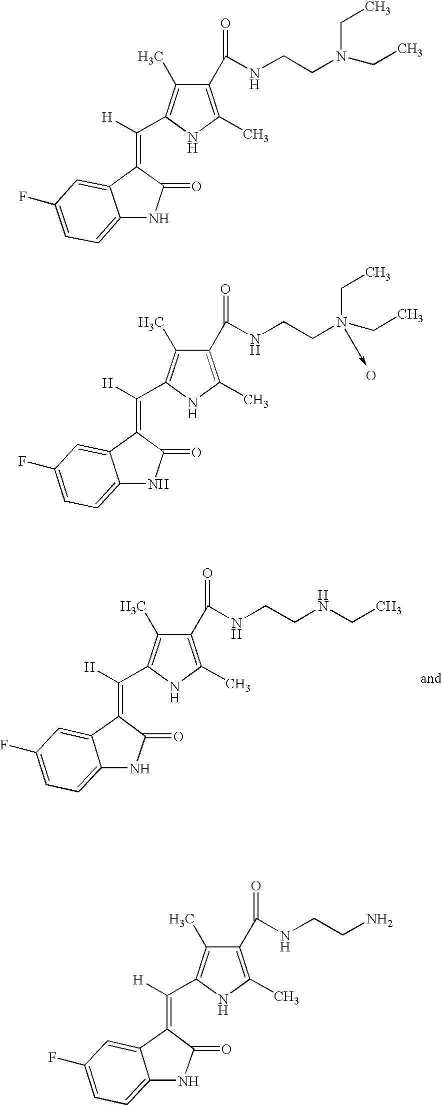

10. The method of claim 1, wherein the amount of CO₂ added is effective to decrease the reaction time t_{1/2} of the compound of formula 1 with the compound of formula 3 to no more than 75% of the reaction time t_{1/2} of the corresponding reaction in the absence of added CO₂.

11. The method of claim 1, wherein the amount of CO₂ added is effective to decrease the reaction time t_{1/2} of the compound of formula 1 with the compound of formula 3 to no more than 60% of the reaction time t_{1/2} of the corresponding reaction in the absence of added CO₂.

12. The method of claim 1, wherein the amount of CO₂ added is effective to decrease the reaction time t_{1/2} of the compound of formula 1 with the compound of formula 3 to no more than 50% of the reaction time t_{1/2} of the corresponding reaction in the absence of added CO₂.

13. The method of claim 1, wherein the reaction of the compound of formula 1 with the compound of formula 3 is carried out in at least one solvent, and at least a portion of the added CO₂ is provided by introducing CO₂ into the solvent.

14. The method of claim 13, wherein the CO₂ is introduced into the solvent before a step of combining the solvent with at least one of the compound of formula 1 and the compound of formula 3.

15. The method of claim 13, wherein the CO₂ is introduced into the solvent after a step of combining the solvent with at least one of the compound of formula 1 and the compound of formula 3.

16. The method of claim 1, wherein at least a portion of the added CO₂ is provided by introducing CO₂ into a reaction mixture comprising the compound of formula 1 and the compound of formula 3.

17. A method of preparing a compound of formula 8 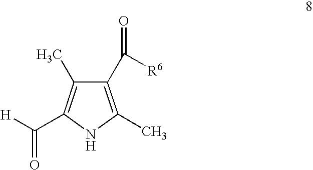
wherein
R⁶ is selected from —NH(CH₂)ₘR⁹ and —NHR¹¹, provided that optionally one to two of the CH₂ groups may be substituted by —OH or halogen;
R⁹ is selected from the group consisting of —NR¹⁰R¹¹, —OH, —C(O)R¹², C₆₋₁₂ aryl, C₆₋₁₂ alkaryl, C₆₋₁₂ aryloxy, C₆₋₁₂ alkaryloxy, C₁₋₁₂ alkoxy, C₂₋₁₂ heterocyclic group containing 1 to 3 atoms selected from N⁻, S and O, —N⁺(O⁻)R¹⁰, and —NHC(O)R¹³;
R¹⁰ and R¹¹ are independently selected from the group consisting of hydrogen, C₁₋₁₂ alkyl, C₁₋₁₂ cyanoalkyl, C₃₋₁₂ cycloalkyl, C₆₋₁₂ aryl, and C₂₋₁₂ heterocyclic group containing 1 to 3 atoms selected from N, S or O; or R¹⁰ and R¹¹ may be combined to form a four-, five- or six-membered heterocyclic group optionally containing 1 to 3 atoms selected from N, O, or S in addition to the nitrogen atom to which R¹⁰ and R¹¹ are bound, provided that the heterocyclic group formed by R¹⁰ and R¹¹ may optionally be substituted by a C₁₋₁₂ alkyl, C₁₋₂ cyanoalkyl, C₅₋₁₂ cycloalkyl, C₆₋₁₂ aryl, or a C₂₋₁₂ heterocyclic group containing 1 to 3 atoms selected from N, S and O;
R¹² is selected from the group consisting of —OH, C₁₋₁₂ alkoxy, C₆₋₁₂ alkaryl and C₆₋₁₂ aryloxy;
R¹³ is selected from the group consisting of C₁₋₁₂ alkyl, C₁₋₁₂ haloalkyl, and C₁₋₁₂ aralkyl; and
m is 1, 2, 3 or 4,
the method comprising reacting a compound of formula 9 with a compound of formula 3 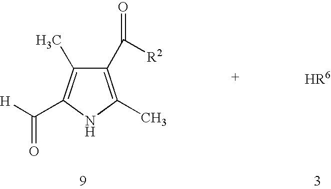
and R is selected from the group consisting of 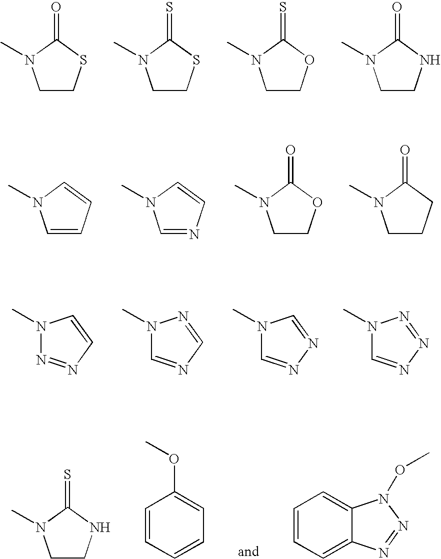
and R² is optionally substituted by 1 to 6 groups independently selected from the group consisting of halogen, C₁₋₆ alkyl, C₁₋₆ alkoxy, C₆₋₁₂ aryl, C₆₋₁₂ aryloxy, C₆₋₁₂ alkaryl, —NHC(O)R¹⁴ and —C(O)OR¹⁴, where R¹⁴ is hydrogen or a C₁₋₄ alkyl,
in the presence of added CO₂, to form the compound of formula 8.

18. The method of claim 17, wherein the step of reacting the compound of formula 9 with the compound of formula 3 comprises:
(i) forming an intermediate of formula 10 if R⁶ is —NH(CH₂)ₘR⁹ or of formula 11 if R⁶ is NHR¹¹ 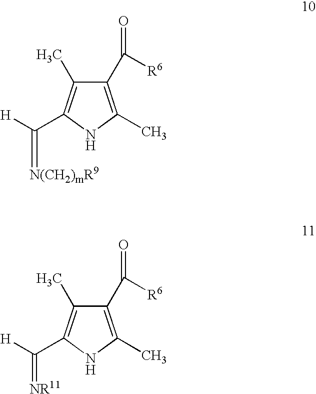
and
(ii) hydrolyzing the imine moiety of the intermediate to form the compound of formula 8.

20. The method of claim 18, wherein R⁶ is selected from the group consisting of —NHCH₂CH₂N(CH₂CH₃)₂, —NHCH₂CH₂NHCH₂CH₃, —NHCH₂CH₂NH₂ and —NHCH₂(C₆H₅).

21. The method of claim 17, further comprising reacting the compound of formula 8 with a compound of formula 6 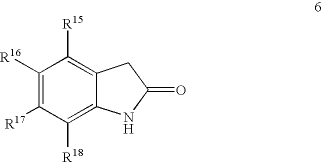
wherein R¹⁵, R¹⁶, R¹⁷ and R¹⁸ are independently selected from the group consisting of hydrogen, C₁₋₁₂ alkyl, C₁₋₂ alkoxy, C₃₋₁₂ cycloalkyl, C₆₋₁₂ aryl, C₂₋₁₂ heterocyclic group containing 1 to 3 atoms selected from N, S and O, C₆₋₁₂ aryloxy, C₆₋₁₂ alkaryl, C₆₋₁₂ alkaryloxy, halogen, trihalomethyl, —S(O)R⁴, —SO₂NR⁴R⁵, —SO₃R⁴, —SR⁴, —NO₂, —NR⁴R⁵, —OH, —CN, —C(O)R⁴, OC(O)R⁴, —NHC(O)R⁴, —(CH₂)ₙCO₂R⁴, and —CONR⁴R⁵;
to form a compound of formula 12

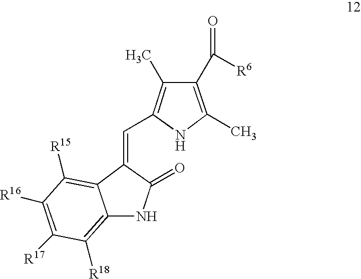

22. The method of claim 21, wherein the compound of formula 12 is selected from the group consisting of

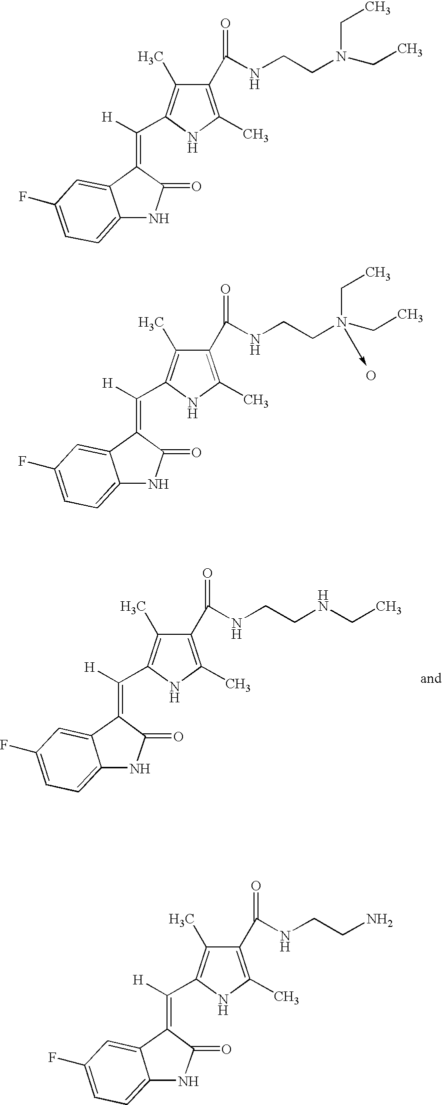

23. The method of claim 17, wherein the amount of CO₂ added is effective to decrease the reaction time t_{1/2} of the compound of formula 9 with the compound of formula 3 to no more than 75% of the reaction time t_{1/2} of the corresponding reaction in the absence of added CO₂.

24. The method of claim 17, wherein the amount of CO₂ added is effective to decrease the reaction time t_{1/2} of the compound of formula 9 with the compound of formula 3 to no more than 60% of the reaction time t_{1/2} of the corresponding reaction in the absence of added CO₂.

25. The method of claim 17, wherein the amount of CO₂ added is effective to decrease the reaction time t_{1/2} of the compound of formula 9 with the compound of formula 3 to no more than 50% of the reaction time t_{1/2} of the corresponding reaction in the absence of added CO₂.

26. The method of claim 17, wherein the reaction of the compound of formula 1 with the compound of formula 3 is carried out in at least one solvent, and at least a portion of the added CO₂ is provided by introducing CO₂ into the solvent.

27. The method of claim 26, wherein the CO₂ is introduced into the solvent before a step of combining the solvent with at least one of the compound of formula 9 and the compound of formula 3.

28. The method of claim 26, wherein the CO₂ is introduced into the solvent after a step of combining the solvent with at least one of the compound of formula 9 and the compound of formula 3.

29. The method of claim 26, wherein at least a portion of the added CO₂ is provided by introducing CO₂ into a reaction mixture comprising the compound of formula 9 and the compound of formula 3.

30. A method of preparing a compound of formula 13 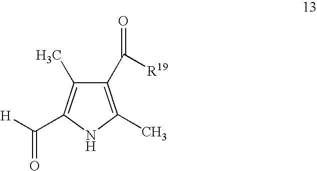
wherein
R¹⁹ is selected from the group consisting of —NHCH₂CH₂N(CH₂CH₃)₂, —NHCH₂CH₂NHCH₂CH₃, —NHCH₂CH₂NH₂ and —NHCH₂(C₆H₅),
the method comprising reacting a compound of formula 14 with a compound of formula 15 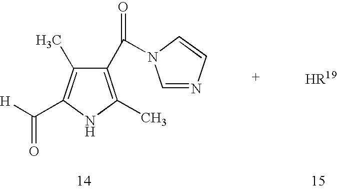
in the presence of added CO₂, to form the compound of formula 13.

31. The method of claim 30, wherein the step of reacting the compound of formula 14 with the compound of formula 15 comprises:
(i) forming an intermediate of formula 16 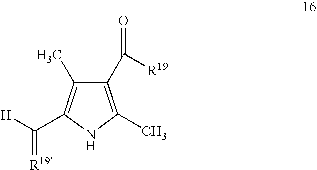
wherein R^{19′} represents an R¹⁹ group with one nitrogen-bound hydrogen removed to accommodate the imine bond; and
(ii) hydrolyzing the imine moiety of the intermediate to form the compound of formula 13.

32. The method of claim 30, further comprising reacting the compound of formula 13 with a compound of formula 17 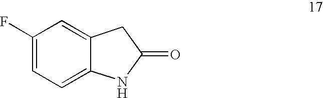
to form a compound of formula 18

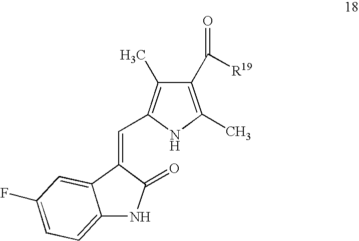

33. The method of claim 30, wherein the amount of CO₂ added is effective to decrease the reaction time t_{1/2} of the compound of formula 14 with the compound of formula 15 to no more than 75% of the reaction time t_{1/2} of the corresponding reaction in the absence of added CO₂.

34. The method of claim 30, wherein the amount of CO₂ added is effective to decrease the reaction time t_{1/2} of the compound of formula 14 with the compound of formula 15 to no more than 60% of the reaction time t_{1/2}, of the corresponding reaction in the absence of added CO₂.

35. The method of claim 30, wherein the amount of CO₂ added is effective to decrease the reaction time t_{1/2} of the compound of formula 14 with the compound of formula 15 to no more than 50% of the reaction time t_{1/2} of the corresponding reaction in the absence of added CO₂.

36. The method of claim 30, wherein the reaction of the compound of formula 14 with the compound of formula 15 is carried out in at least one solvent, and at least a portion of the added CO₂ is provided by introducing CO₂ into the solvent.

37. The method of claim 36, wherein the CO₂ is introduced into the solvent before a step of combining the solvent with at least one of the compound of formula 14 and the compound of formula 15.

38. The method of claim 36, wherein the CO₂ is introduced into the solvent after a step of combining the solvent with at least one of the compound of formula 14 and the compound of formula 15.

39. The method of claim 36, wherein at least a portion of the added CO₂ is provided by introducing CO₂ into a reaction mixture comprising the compound of formula 14 and the compound of formula 15.

40. A compound of formula 20 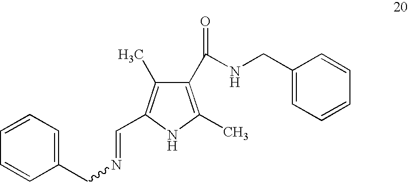
or a salt or hydrate thereof.

41. A compound of formula 21 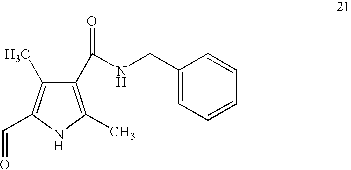
or a salt or hydrate thereof.

42. A compound of formula 22 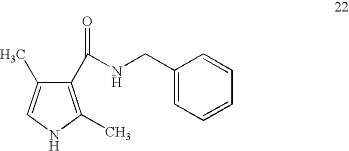
or a salt or hydrate thereof.

43. A compound of formula 23 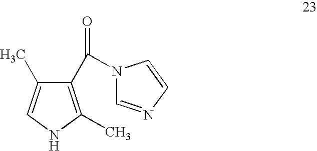
or a salt or hydrate thereof.
